(19) United States
(12) Patent Application Publication
(10) Pub. No.: US 2015/0164498 A1
(43) Pub. Date: Jun. 18, 2015

(54) SUTURE WASHER

(71) Applicant: Arthrex, Inc., Naples, FL (US)

(72) Inventors: Peter J. Dreyfuss, Naples, FL (US); Benjamin Chan, Naples, FL (US); John A. Sodeika, Naples, FL (US); Matthew T. Provencher, Boston, MA (US)

(21) Appl. No.: 14/567,512

(22) Filed: Dec. 11, 2014

Related U.S. Application Data
(60) Provisional application No. 61/914,586, filed on Dec. 11, 2013.

(51) Int. Cl. A61B 17/04; A61L 17/14; A61B 17/06

(52) U.S. Cl. CPC A61B 17/0401; A61B 17/06166; A61L 17/145; A61B 2017/0464; A61B 2017/0445; A61B 2017/0414; A61L 2420/00

(57) ABSTRACT

A suture washer construct for fixation of soft tissue to bone, or of soft tissue to soft tissue. The suture washer provides enhanced fixation and soft tissue protection. The washer consists of a cannulated metal washer with a series of through passages running around the body of the washer, to allow one or more flexible strands (for example, one or more sutures) to extend through the passages. The washer is threaded over a fixation device (screw or anchor) before insertion. Once the fixation device (screw or anchor) is inserted, the surgeon has the ability to pass the flexible strand (suture) as with a typical anchor.

### CROSS-REFERENCE TO RELATED APPLICATIONS

This application claims the benefit of U.S. Provisional Application No. 61/914,586, filed Dec. 11, 2013, the entire disclosure of which is incorporated by reference herein.

### FIELD OF THE INVENTION

The present invention relates to the field of surgery and, more particularly, to improved methods of incorporating suture or similar materials in orthopedic procedures.

### BACKGROUND OF THE INVENTION

Fixation devices such as screws are often used in orthopedic procedures to stabilize the bone and facilitate tissue healing. Often, it is necessary to incorporate suture in these orthopedic repairs for additional fixation of soft tissue. Incorporation of the suture with the fixation devices (for example, screws) could be difficult, however, as the incorporation requires additional steps and it is often time consuming.

For example, in order to complete a Latarjet procedure, the surgeon must first dissect away the capsule to expose the bony glenoid surface. The surgeon will then screw the allograft into place using screws, typically stainless steel or titanium screws. After the graft is in place, the surgeon must re-attach the capsule to bone. This is typically done with additional sutures and plastic anchors. This operation adds cost, steps, and time to the overall procedure.

To reduce the amount of time and number of implants, some surgeons will attempt to re-attach the capsule by simply passing suture through tissue and then tying the suture around the screw heads. This method is much faster but it runs the risk of the sutures unraveling from around the screw head. Additionally, once the suture is wrapped around the screw, the surgeon loses the ability to slide the suture in order to orient and readjust the suture in the most biomechanically favorable way.

Improved surgical implements that would allow the user (surgeon) to use a suture during a surgical orthopedic procedure with the ability to slide the suture, and to allow for easy orientation of the suture relative to the bone are needed. Also needed are improved methods of surgical tying and suture manipulation with decreased suture tear and increased fixation. Improved Latarjet procedures with reduced number of surgical steps and reduced OR time are also needed.

### SUMMARY OF THE INVENTION

The present invention provides methods and suture constructs including a suture washer construct for fixation of soft tissue to bone, or of soft tissue to soft tissue.

Fixation of soft tissue to bone (or of soft tissue to soft tissue) is conducted using a washer (for example, a suture washer) that provides enhanced fixation and soft tissue protection. The washer consists of a cannulated metal washer with a series of through holes running around the body of the implant, to allow one or more flexible strands (for example, one or more sutures) to extend through the holes. The washer is attached to a fixation device (for example, a screw or anchor) before insertion of the fixation device. Once the fixation device is inserted, the surgeon has the ability to pass the flexible strand (suture) as with a typical anchor.

The suture washer construct of the present invention reduces the number of steps required to perform the Latarjet procedure and reduces the OR time. The suture washer of the present invention also addresses all the above-mentioned problems associated with fixation of soft tissue during orthopedic procedures (i.e., eliminates the risk of the suture unraveling from the screw head, allows for easy orientation of the suture in the desired position, reduces the number of implants required for insertion, decreases the risk of bone fracture, etc.).

These and other features and advantages of the present invention will become apparent from the following description of the invention that is provided in connection with the accompanying drawings and illustrated embodiments of the invention.

### BRIEF DESCRIPTION OF THE DRAWINGS

FIG. 1(a) illustrates a top view of a washer with holes in accordance with an exemplary embodiment of the present invention (depicting a 3.75 mm titanium screw with three exemplary holes).

FIG. 1(b) illustrates a cross-sectional view of the washer of FIG. 1(a), taken along line B-B.

FIG. 1(c) illustrates a cross-sectional view of the washer of FIG. 1(a), taken along line A-A.

FIG. 1(d) illustrates another cross-sectional view of the washer of FIG. 1(a), taken along line A-A.

FIGS. 2(a) and 2(b) illustrate perspective and top views of another exemplary embodiment of a washer of the present invention.

FIG. 3 illustrates another exemplary embodiment of a washer of the present invention.

FIG. 4 illustrates another exemplary embodiment of a washer of the present invention.

FIG. 5 illustrates a perspective view of a washer/flexible strand/needle assembly of the present invention, depicting the washer of FIG. 1(a) with a flexible strand passed therethrough and attached to an exemplary needle.

FIGS. 6-13 illustrate subsequent steps of a Latarjet procedure utilizing the washer of FIG. 1(a) attached to an exemplary fixation device (screw) and according to an exemplary embodiment of the present invention.

### DETAILED DESCRIPTION OF THE PREFERRED EMBODIMENTS

The present invention provides methods and constructs for fixation of soft tissue to bone which protect the soft tissue and preserve the bone mass with an attached suture washer construct. The present invention provides methods and suture constructs including a suture washer for fixation of soft tissue to bone.

Fixation of soft tissue to bone (or of soft tissue to soft tissue) is conducted using a washer (for example, a suture washer) that provides enhanced fixation and soft tissue protection. The washer consists of a cannulated metal washer with a series of through holes running around the body of the implant, to allow one or more flexible strands (for example, one or more sutures) to extend through the holes. The washer is attached to a fixation device (for example, a screw or anchor) before insertion of the fixation device. The washer may be attached by threading it over the fixation device (for example, over the screw). Once the fixation device is inserted, the surgeon has the ability to pass the flexible strand (suture) as with a typical anchor.

The present invention also provides a washer/flexible strand/needle construct consisting of a cannulated metal washer provided with a series of through passages running around the body of the washer; at least one flexible strand (for example, at least one suture) extending through the passages of the cannulated metal washer and exiting the cannulated metal washer through two apertures/orifices located on different regions of an outer surface of the washer; and a needle attached to the at least one flexible strand (for example, attached by crimping or other known methods in the art). The washer/flexible strand/needle construct may be provided pre-packaged and pre-assembled and optionally with additional fixation devices (for example, bone screws and/or anchors) to aid in the fixation of a first tissue to a second tissue, and as detailed below.

The present invention also provided methods of fixation of first tissue to a second tissue by inter alia the steps of: (i) providing a washer consisting of a cannulated metal washer with a series of through holes/passages running around the body of the washer, and with at least one flexible strand (for example, at least one suture) extending through the holes/passages; (ii) securing the washer to a fixation device (for example, a screw or anchor); (iii) inserting the fixation device with the secured washer into a first tissue; (iv) passing the at least one flexible strand through or around a second tissue; and (v) orienting and readjusting the at least one flexible strand, with decreased flexible strand tear and increased fixation. In an exemplary embodiment, the washer is a suture washer and the at least one flexible strand is suture.

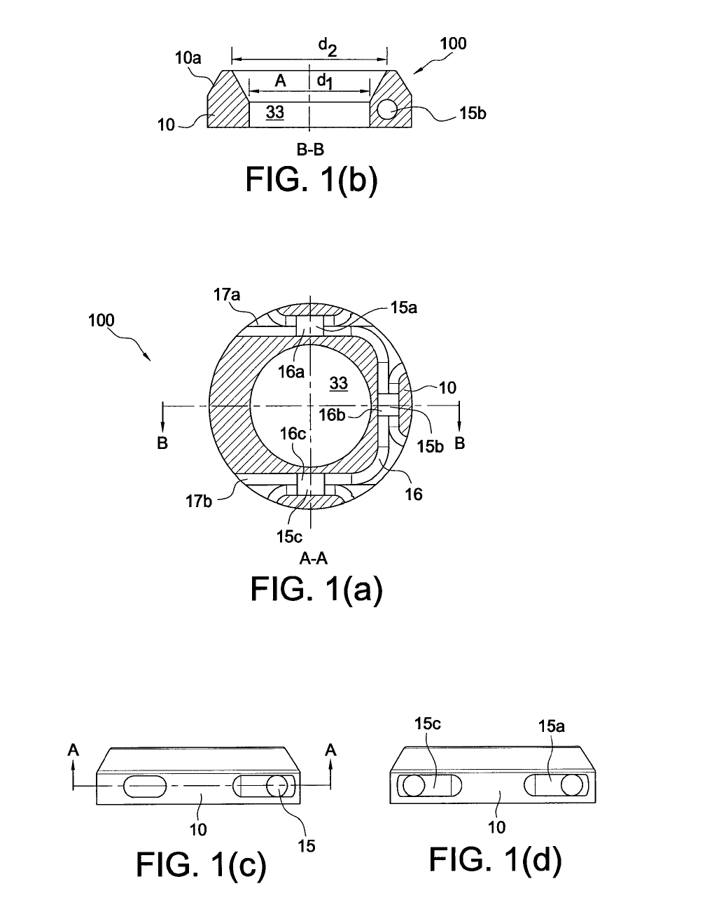

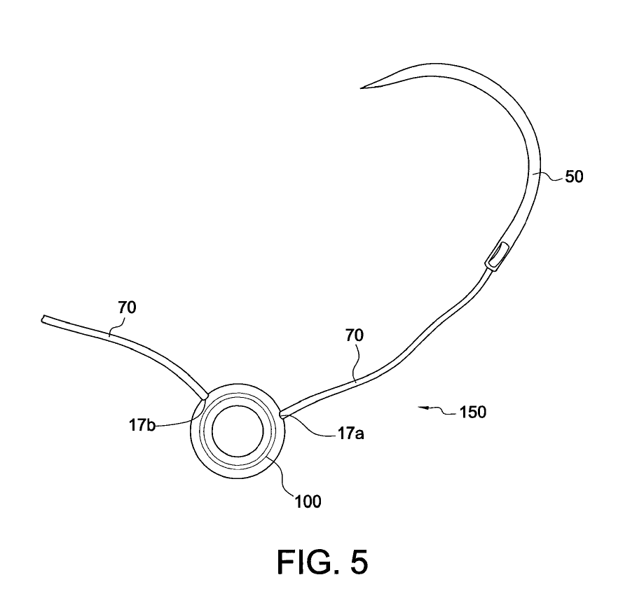

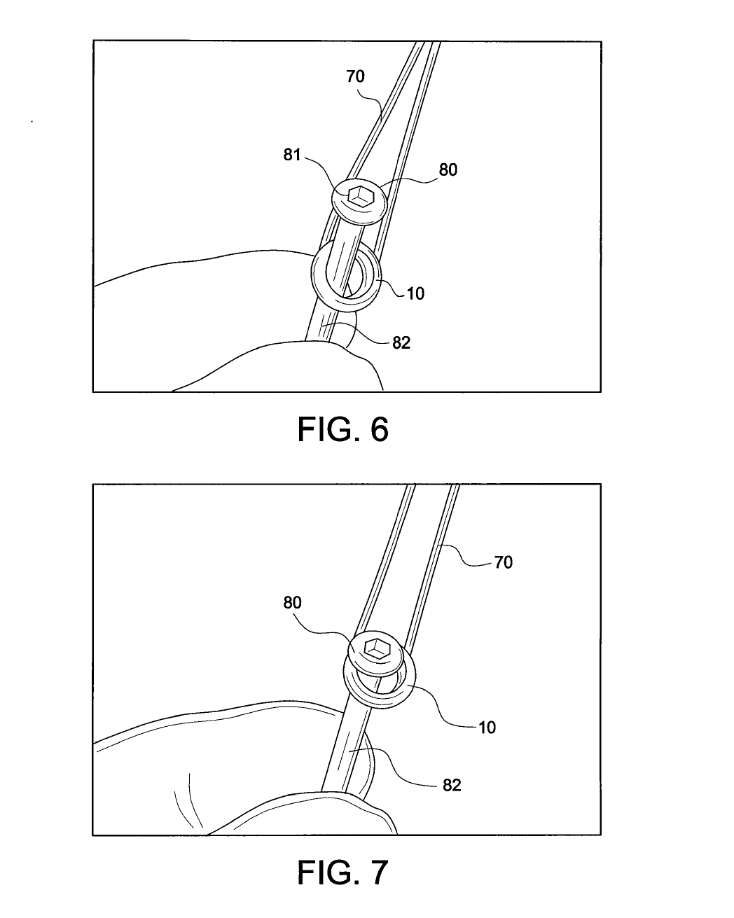

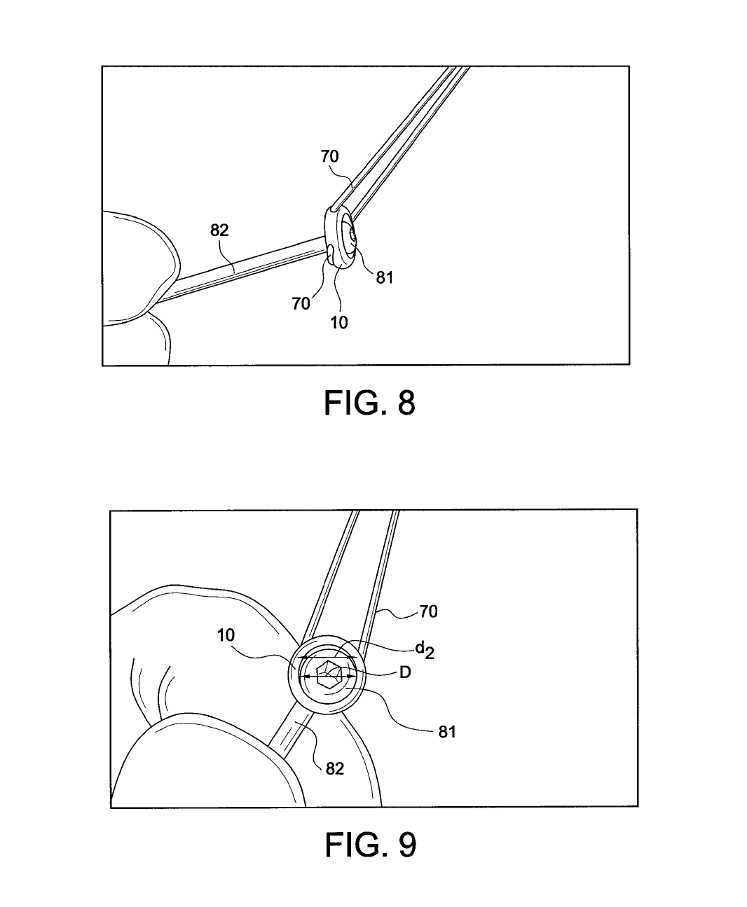

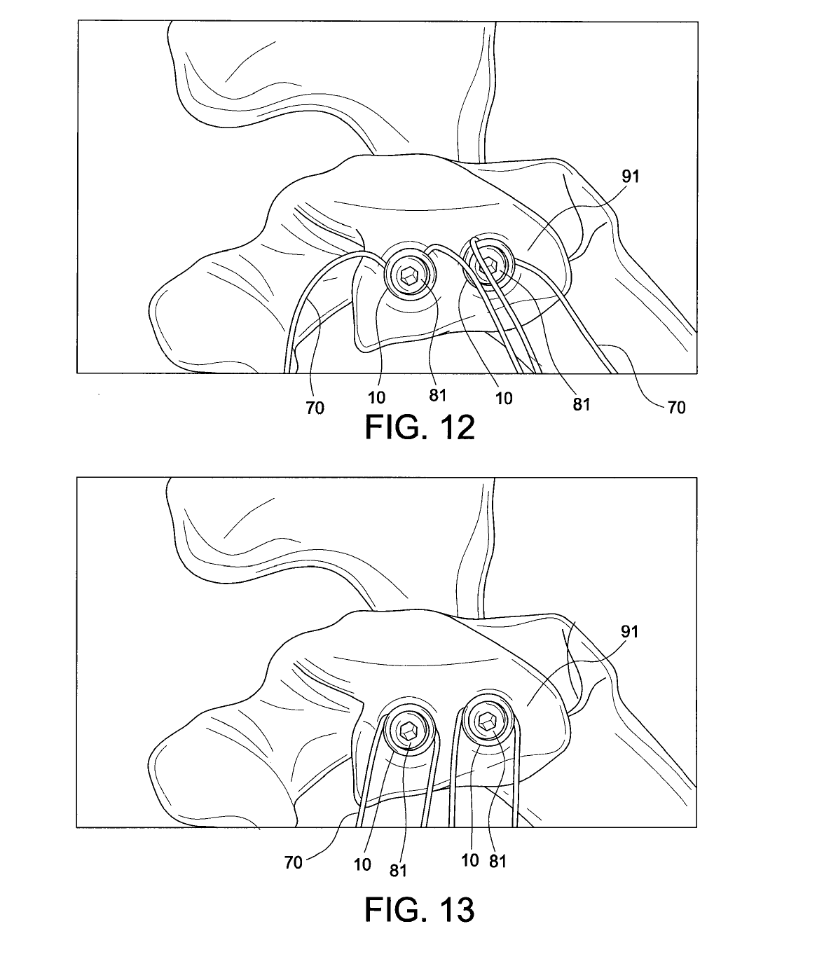

Referring now to the drawings, where like elements are designated by like reference numerals, FIGS. 1(a)-4 illustrate exemplary washers 100, 200, 300, 400 of the present invention. FIG. 5 illustrates an exemplary washer/suture/needle assembly 150 of the present invention incorporating exemplary washer 100 of FIG. 1(a). FIGS. 6-13 illustrate exemplary subsequent steps of a Latarjet method that employs exemplary washer 100 of the present invention.

Although reference to washer 100, 200, 300, 400 will be made below as to suture washer 100, 200, 300, 400, the invention is not limited to this exemplary-only embodiment and incorporates washers that allow any flexible strand to slide therethrough and, thus, not limited to suture. The washers described below also allow one or more flexible strands (which could be made of similar or different materials) to easily slide and pass therethrough.

Exemplary suture washer 100 shown in FIGS. 1(a)-(d) is a cannulated metal washer formed of body 10 provided with a series of through inner holes or passages 15a, 15b, 15c running around the body 10 of the washer 100 and in communication with hole 16 having a “U” shape (as shown in FIG. 1(a)). Inner passages 15a, 15b, 15c allow one or more flexible strands 70 (for example, one or more sutures or suture tapes 70) to extend through them and to freely move within the body 10 of the washer. In an exemplary-only embodiment, washer 100 is provided with three inner suture holes or passages 15a, 15b, 15c that have a “C” shape configuration (as shown in FIG. 1(a)); however, the invention contemplates suture washers with any number of suture holes or passages, which may have similar and/or different configurations. The inner passages have a cylindrical cross-section and are provided with a diameter at least twice greater than the diameter of the flexible strand that passes through the passages, to allow the flexible strand to easily slide therethrough. Preferably, all inner passages 15a, 15b, 15c have an equal diameter.

Passages/holes 15a, 15b, 15c have a common area 16a, 16b, 16c with “U” shaped hole or passage 16 (i.e., extend along at least a portion of “U” shaped hole 16) to allow a flexible strand to freely slide/pass through the washer and allow secure attachment of first tissue to second tissue (for example, soft tissue to bone). In an exemplary-only embodiment, “U” shaped passage 16 extends within the body 10 of washer 100 for about ¾ the circumference of the body, and from a first aperture or orifice 17a (provided within a first area of outer surface 10a of body 10) to a second aperture or orifice 17b (provided within a second area of outer surface 10a of body 10).

Washer 100 comprises body 10 that is adapted to engage a bone anchor or bone screw while also allowing a flexible strand to pass through a cannulation of the body. Body 10 may have a disc-shaped or donut-shaped configuration defining a central aperture 33 or a screw-retaining aperture 33 (having stepped inner diameter) and dimensioned to receive and accept the head of a bone screw or anchor. The screw-retaining aperture 33 is adapted to allow insertion of a bone screw through the bone screw-retaining aperture 33 and positioning of the washer against a head of the bone screw, as detailed below.

Body 10 may be formed of a metal, preferably titanium or titanium alloy, or other biocompatible metals or compositions known in the art.

As shown more clearly in FIG. 1(b), the inner diameter of the body of the suture washer 100 is a stepped diameter, ranging from a small diameter “d1” to a largest diameter “d2” of the body of the washer, to preserve material and allow secure engagement to a fixation device, for example, a head of a screw, as detailed below with reference to FIGS. 6-13. Diameter “d2” of the body 10 is greater than the outer diameter D of head 81 of fixation device 80 (as shown in FIG. 9, for example) to allow the head of the fixation device to rest within area A of the body 10 (FIG. 1(b)).

The suture washer 100 is designed to be threaded over a fixation device (for example, a bone screw or anchor) before insertion. Once the fixation device (screw) is inserted, the surgeon has the ability to pass the flexible strand 70 (suture 70) as with a typical anchor. The washer may be flexibly secured to a first tissue (for example, bone) by means of the fixation device (bone screw or anchor)

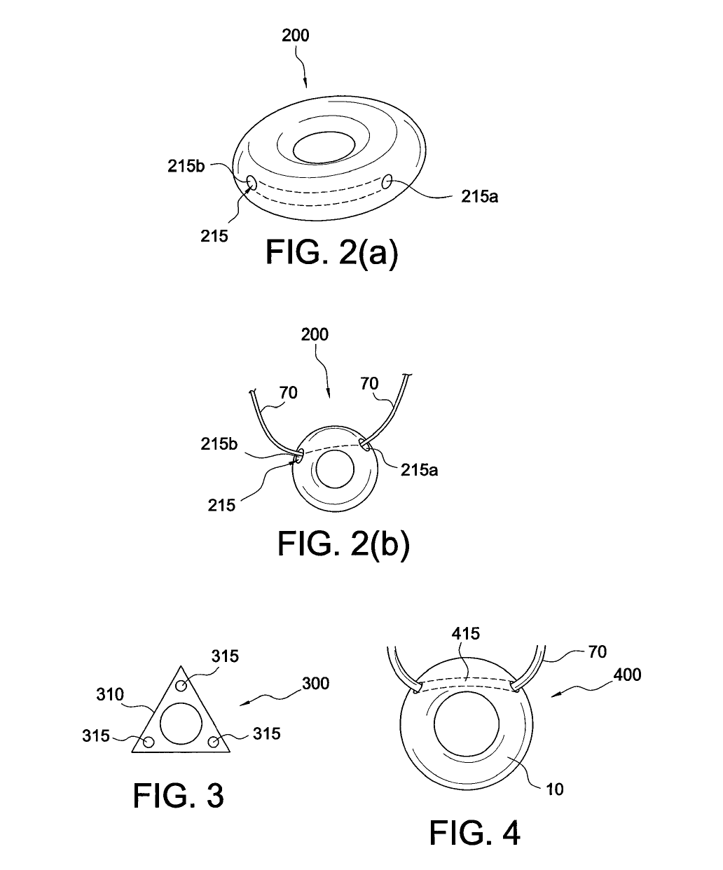

FIGS. 2(a)-4 illustrate additional embodiments of suture washers 200, 300, 400 of the present invention. For example, FIGS. 2(a) and 2(b) illustrate suture washer 200 which is provided with a single straight hole or passage 215 that has a linear, straight configuration and passes through body 10 of the suture washer, allowing flexible strand 70 to enter orifice/aperture 215a and exit orifice/aperture 215b, as shown in FIG. 2(b), for example. Suture washer 300 shown in FIG. 3 has body 310 with a general triangular configuration and with exemplary holes or apertures 315 provided in each corner of the triangular body 310. Suture washer 400 of FIG. 4 is also a circular washer provided with a straight linear hole or passage 415 that allows one or more strands 70 to freely slide and pass therethrough.

Although washer 400 is about similar to washer 100, washer 400 is different in that it has a larger diameter than washer 100, to allow passage of wider flexible materials/strands, for example, labral tape, FiberTape® or TigerTape, or similar flexible strands and wider materials, and to be accommodated by larger fixation devices, for example, by screws with diameters between about 3.5 mm to about 6.5 mm.

The small diameter washers could be cylindrical with a 2.0 mm diameter to accommodate and engage an exemplary fixation device (screw) with a diameter of about 3.5/4.0 mm, for example, a 3.7 mm glenoid screw.

Suture washers 100, 200, 300, 400 may be used for soft tissue reattachment whenever screws or similar fixation devices are currently used, for example, fracture fixation, arthrodesis and osteotomy, among many others. Suture washers 100, 200, 300, 400 have bodies with various configurations and/or geometries that permit attachment to various fixation devices such as screws, anchors, etc. If desired, and depending on the specific surgical application, washers 100, 200, 300, 400 may be also provided with spikes or similar structures. Washer 200 illustrates, for example, an exemplary design for anterior capsular repair with fixation devices (screws) in anterior instability. Suture washers 100, 200, 300, 400 may be employed, however, in a variety of surgical applications, for example, fracture repairs, arthrodesis, osteotomies, knee ligament repairs, elbow ligament/tendon repairs or fixations, rotator cuff repairs, Achilles tendon repair, patellar tendon repair, ACL repair as a back-up washer, among many others.

FIG. 5 illustrates exemplary washer 100 of FIG. 1(a) assembled/provided with flexible strand 70 attached to a passing instrument, for example, curved needle 50, to form flexible strand/washer/needle assembly 150. The flexible strand and needle may be provided pre-assembled or pre-packaged with the washer 100, in the form of a kit or similar assembly. The needle may be straight or curved and may be releasably securable to the flexible strand.

The suture washer construct 100, 200, 300, 400 of the present invention reduces the number of steps required to perform the Latarjet procedure and reduce the OR time. The suture washer construct 100, 200, 300, 400 also addresses all the above-mentioned problems associated with fixation of soft tissue during orthopedic procedures. First, the suture washer 100, 200, 300, 400 allows for a neat repair and eliminates the risk of the suture unraveling from the screw head. The inner diameter of the washer is less than that of the screw head, meaning that the capsule cannot detach from the bony surface without pulling out the screw, or having the suture rip through the tissue. The suture washer 100, 200, 300, 400 eliminates the need for additional suture anchors, and soft tissue to bony fixation can be incorporated into the first steps of the repair.

Second, the suture 70 can slide within the holes 15a, 15b, 15c of the suture washer 100, 200, 300, 400, and the construct allows for easy orientation of the suture 70 whichever way the surgeon chooses. This aspect reduces the difficulty and time required to complete the overall surgical repair, which contribute to better patient outcomes.

Third, the suture washer 100, 200, 300, 400 also reduces the number of implants required for insertion. Each hole drilled into the bone reduces the overall strength of the bone and of the repair. Each additional suture anchor implanted increases the number of holes created in the bone (for example, glenoid) which, in turn, increases the chance of fracture. The suture washer 100, 200, 300, 400 allows for the metal screws to double as a suture anchor, which reduces the number of holes created during the procedure.

Fourth, the suture washer 100, 200, 300, 400 allows for the suture 70 to be loaded over a much broader area. When wrapping the suture around the screw head, or point loading it within an anchor, the action puts uneven stress on the suture which can lead to premature failure. The suture washer 100, 200, 300, 400 allows for the suture 70 to be loaded evenly across the length within the washer, which can potentially prevent suture breakage.

The suture washers 100, 200, 300, 400 of the present invention are not provided with any spikes or protuberances to facilitate locking the orientation of the washer in place, along with holes oriented to allow the suture to be compressed against the bone and the washer. Instead, the suture washers 100, 200, 300, 400 of the present invention have a design that is superior in that the cannulation in the washer allows for the suture to slide freely. This increases the ease in which the sutures can be manipulated and subsequently passed through tissue. Additionally, the suture washers 100, 200, 300, 400 of the present invention have no spikes that control the orientation of the washer. This allows for last minute adjustments and also allows for the washer to be oriented such that the suture has less possibility to rub against the edge of the implant, reducing the chance of suture breakage.

Suture washers 100, 200, 300, 400 may be employed for any fixation of first tissue to second tissue, for example, for any fixation of soft tissue or capsular tissue to bone. An exemplary method of fixation of first tissue to a second tissue according to an embodiment of the present invention comprises inter alia the steps of: (i) providing a washer 100, 200, 300, 400 consisting of a cannulated metal washer with a series of through holes running around the body of the washer, and with one or more flexible strands 70 (for example, one or more sutures) extending through the holes; (ii) threading the washer 100, 200, 300, 400 over a fixation device 80 (for example, screw) and securing the washer to an element 81 of the fixation device 80; (iii) inserting the fixation device (screw) with the secured washer into a first tissue 91; (iv) passing the one or more flexible strands through or around a second tissue 93; and (v) orienting and readjusting the one or more flexible strands 70, with decreased flexible strand tear and increased fixation.

An exemplary Latarjet method for shoulder dislocation or subluxation with suture washer 100 of FIG. 1(a) is shown in FIGS. 6-13. Washer 100 provided with flexible strand 70 is threaded over the fixation device 80 (screw) before insertion (FIG. 6). The washer is brought up the screw stem/shank 82 and adjacent and in contact with head 81 of screw 80 (FIGS. 7-9). Flexible strand 70 can easily slide along the circumference of washer 100.

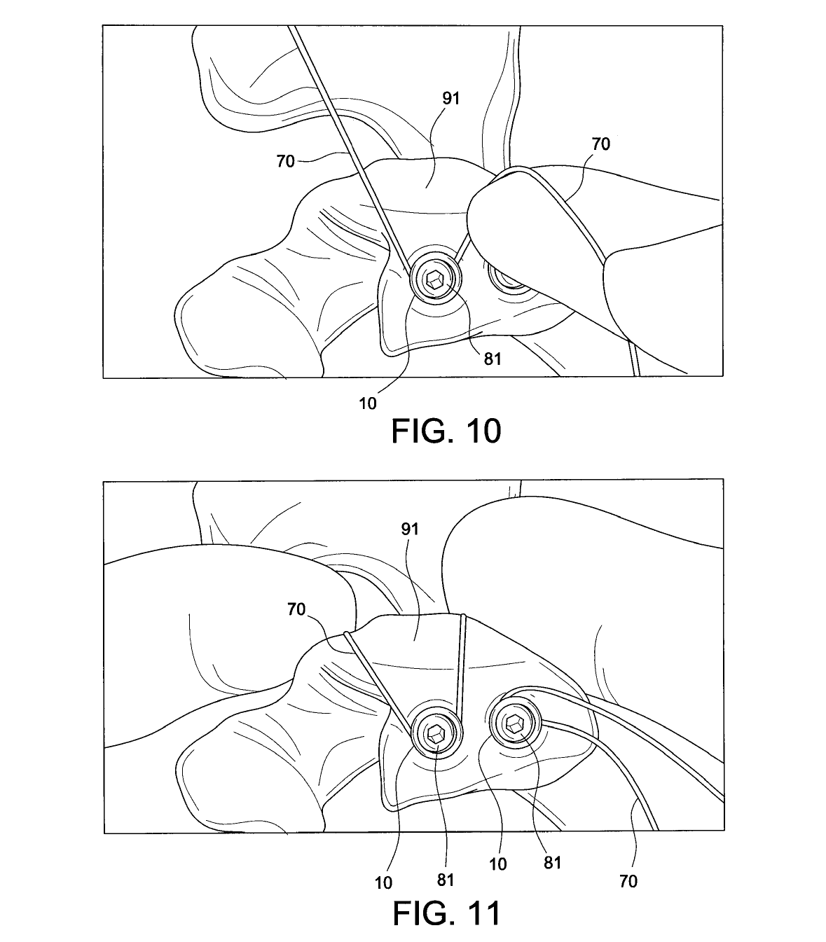

FIGS. 10-13 show two fixation devices 80 with two washers 100 inserted into bone 91. Once the fixation devices 80 (screws 80) are inserted, the surgeon has the ability to pass the flexible strand 70 (suture 70) as with a typical anchor. Suture 70 may be attached to a second tissue, for example, capsular tissue or allograft, to complete the repair.

The one or more flexible strands 70 employed with the washers 100, 200, 300, 400 of the present invention may include sutures, braids, tapes, suture chains, suture tapes, sutures with collagen, or combinations thereof. In an exemplary and illustrative embodiment only, at least one of the flexible strands 70 is a suture strand formed of ultrahigh molecular weight polyethylene (UHMWPE) such as FiberWire® suture. The flexible strand 70 may also contain a bioabsorbable material, such as PLLA or one of the other polylactides, for example, and/or may be formed of twisted fibers having strands of a contrasting color added to the braided threads, to make the suture more visible during surgical procedures.

The one or more flexible strands 70 may be provided with or without needles. The needles may be attached to the flexible strand/suture by any known method in the art, for example, by being swedged or crimped onto the suture. If a kit is provided, the kit may include a plurality of washer/suture/needle constructs 150 depending on the characteristics of the tissue to be repaired/attached.

The flexible strand 70 may be any material strand, for example, suture, suture tape such as FiberTape®, suture chain such as FiberChain®, or any flexible material.

The flexible strand 70 may include a high-strength suture, such as an ultrahigh molecular weight polyethylene (UHMWPE) suture. Alternatively, the high strength suture may be a FiberWire® suture, which is disclosed and claimed in U.S. Pat. No. 6,716,234, the entire disclosure of which is incorporated herein by reference. FiberWire® suture is formed of an advanced, high-strength fiber material, namely ultrahigh molecular weight polyethylene (UHMWPE), sold under the tradenames Spectra (Honeywell) and Dyneema (DSM), braided with at least one other fiber, natural or synthetic, to form lengths of suture material. The preferred FiberWire® suture includes a core within a hollow braided construct, the core being a twisted yarn of UHMWPE.

The fixation devices/implants/screws/anchors 80 may have various sizes (various diameters and/or lengths) and may be formed of biocompatible materials such as PEEK, biocomposite materials, metals and/or metal alloys, or combination of such materials, among others. The fixation devices/implants/screws/anchors may be also formed of any rigid medically approved materials, for example, plastic or carbon fiber, or combination of different materials.

The flexible strand 70 may be coated (partially or totally) with wax (beeswax, petroleum wax, polyethylene wax, or others), silicone (Dow Corning silicone fluid 202A or others), silicone rubbers (Nusil Med 2245, Nusil Med 2174 with a bonding catalyst, or others) PTFE (Teflon, Hostaflon, or others), PBA (polybutylate acid), ethyl cellulose (Filodel) or other coatings such as collagen, to improve lubricity of the final suture construct, knot security, pliability, handleability or abrasion resistance, for example.

At least one portion of flexible strand 70 may be also provided as a stiff portion to allow the flexible strand/suture to be pushed through the cannulation of the washer and/or through additional suture passing instruments (such as Lasso instruments, for example), if necessary.

Washer 100 may have cross-sections of various forms and geometries, including round, oval, rectangular, or flat, among others, or combination of such forms and geometries, as long as they allow secure engagement and attachment to a fixation device. The diameter of washer 100 may be constant or may vary.

While the present invention is described herein with reference to illustrative embodiments for particular applications, it should be understood that the invention is not limited thereto. Those having ordinary skill in the art and access to the teachings provided herein will recognize additional modifications, applications, embodiments and substitution of equivalents within the scope of the invention. Accordingly, the invention is not to be considered as limited by the foregoing description.

What is claimed is:

1. A surgical washer comprising:
a cannulated washer body provided with at least one passage or hole extending through the cannulated washer body; and
at least one flexible strand extending through the at least one passage or hole.

2. The surgical washer of claim 1, wherein the cannulated washer body has a circular configuration with three different inner passages.

3. The surgical washer of claim 2, wherein each of the three inner passages has a letter “C” shape.

4. The surgical washer of claim 1, wherein the at least one flexible strand is a suture, a suture tape or a suture chain.

5. The surgical washer of claim 1, wherein the at least one flexible strand is a suture with a stiffness that allows the suture to be pushed through a cannulation of the cannulated washer body.

6. The surgical washer of claim 1, wherein the at least one flexible strand is a suture coated with a coating.

7. The surgical washer of claim 6, wherein the coating is a silicon or collagen coating.

8. The surgical washer of claim 1, wherein the at least one flexible strand passes through three different inner passages of the surgical washer.

9. The surgical washer of claim 1, wherein the washer body further comprises a central aperture that is dimensioned to engage and accept a head of a bone screw.

10. A suture washer consisting of a surgical suture and a washer, wherein the washer has a cannulated circular body and is provided with at least one through passage extending from one surface of the washer to another surface of the washer, to allow the surgical suture to pass thereto.

11. The suture washer of claim 10, wherein the circular body has a central aperture extending in a direction about perpendicular to the at least one through passage, and wherein the central aperture has a stepped inner diameter to allow the washer to engage and contact a head of a bone screw.

12. A washer/flexible strand/fixation device assembly comprising:
a washer having a cannulated circular body and provided with at least one through passage extending from a first area of an outer surface of the washer to a second area of the outer surface of the washer;
at least one surgical suture passing through the least one through passage and around the body of the washer; and
a knotless fixation device secured to the washer.

13. The assembly of claim 12, wherein the knotless fixation device is a fixation screw or an anchor.

14. The assembly of claim 13, wherein an inner diameter of the washer is smaller than an outer diameter of the fixation screw or the anchor.

15. The assembly of claim 12, wherein the at least one through passage has a letter “C” shape.

16. The assembly of claim 12, wherein the at least one through passage has a linear straight configuration or a curvilinear configuration.

17. A method of fixation of first tissue to second tissue, the method comprising the steps of:
providing a surgical washer comprising a circular body and at least one passage or hole running around the circular body to allow at least one flexible strand to pass through the at least one passage or hole, and at least one flexible strand passing through the at least one passage or hole;
securing the surgical washer to a fixation device;
securing the fixation device into a first tissue; and
subsequently, attaching the at least one flexible strand to a second tissue.

18. The method of claim 17, wherein the fixation device is a screw or an anchor.

19. The method of claim 17, wherein the first tissue is bone and the second tissue is soft tissue.

20. The method of claim 17, wherein the at least one passage or hole has a letter “C” shape or a straight linear configuration.
